(19) United States
(12) Patent Application Publication
(10) Pub. No.: US 2007/0239043 A1
(43) Pub. Date: Oct. 11, 2007

(54) Method and Apparatus for Arrhythmia Episode Classification

(76) Inventors: Amisha S. Patel, Maple Grove, MN (US); James D. Webb, Maple Grove, MN (US); Bruce D. Gunderson, Plymouth, MN (US); Mark L. Brown, North Oaks, MN (US); Dan B. Carlson, Shoreview, MN (US)

Correspondence Address: MEDTRONIC, INC., 710 MEDTRONIC PARKWAY NE, MINNEAPOLIS, MN, 55432-9924, US

(21) Appl. No.: 11/278,001

(22) Filed: Mar. 30, 2006

(51) Int. Cl. A61B 5/02

(52) U.S. Cl. 600/508; 600/515

(57) ABSTRACT

Apparatus and methods are provided for analyzing an episode stored by an implantable medical device (IMD) using prior probability and conditional probability information to determine the likelihood of a particular diagnosis for a given stored episode. Certain embodiments include retrieving information about a stored episode from an IMD, including an episode metric, and retrieving domain expert information about potential diagnoses and episode metrics to determine the likelihood that the stored episode was due to a particular potential diagnosis. Certain embodiments also include retrieving patient information including a patient metric, such as patient demographics, or patient history. Certain embodiments of the invention include the ability to automatically or manually update or change the domain expert information.

### FIELD OF THE INVENTION

The present invention relates generally to medical devices, and more particularly relates to implantable medical devices (IMDs).

### BACKGROUND OF THE INVENTION

In current clinical practice, when a patient with an implantable medical device (IMD) presents with stored episodes, a clinician/physician must analyze and interpret the stored episode data to determine the patient's condition leading to detection of the episode. Typically, this is done to determine whether the episode was appropriately detected, and/or to determine whether any therapy delivered by the IMD was appropriate and/or effective. For example, a patient with an implantable cardioverter defibrillator (ICD) may present with stored episode data, which may be retrieved and analyzed by a clinician/physician to determine whether the ICD correctly identified and/or classified a cardiac arrhythmia episode.

When analyzing stored episode data, the clinician/physician may typically evaluate a number of different types of information, including information about a given episode (e.g., information specific to a particular episode retrieved from an IMD), information about the particular patient (e.g., demographic information about the patient, or the patient's arrhythmia history), and possibly statistical information from which certain estimates and inferences may be made, for example. Without information about a particular episode, a clinician/physician might know (or estimate) that a certain percentage of detected episodes in a given patient population (in all ICD patients, for example) are actually due to a particular cause (e.g., ventricular tachycardia, or VT). When presented with additional information regarding a specific episode, the clinician/physician may adjust the likelihood of the episode being due to a certain cause accordingly. For example, a clinician/physician analyzing a stored episode from an ICD patient may note that, just prior to detection of the episode, the following episode characteristics are observed: a ventricular cycle length (VCL) that is regular (i.e., having relatively little variability), an atrial/ventricular (A/V) ratio that is 1:1, and stable PR intervals. The clinician/physician, or other “experts” in the field, might tell you that, based on this additional episode-specific information, the probability that the detected rhythm is due to sinus tachycardia (ST), or due to atrial tachycardia (AT) are now much higher, while the probability that it is 1:1 ventricular tachycardia (VT) with retrograde (V-A) conduction is now much less than it was prior to obtaining the additional information. Additional information about the episode, or about the patient, may further affect the probabilities associated with each of a number of potential causes of the episode.

### BRIEF SUMMARY OF THE INVENTION

In certain embodiments of the invention, a method is provided to analyze stored episode information from an implantable medical device (IMD), and determine the likely probability of potential diagnoses. In certain further embodiments of the invention, a method may further determine the most likely diagnosis or cause of detection of the episode by determining the diagnosis with the highest probability.

### BRIEF DESCRIPTION OF THE DRAWINGS

The present invention will hereinafter be described with reference to the following drawing figures, wherein like numerals denote like elements:

FIG. 1 is a schematic diagram depicting a multi-channel, atrial and bi-ventricular, monitoring/pacing implantable medical device (IMD) in which embodiments of the invention may be implemented;

FIG. 2 is a simplified block diagram of IMD circuitry and associated leads that may be employed in the system of FIG. 1 to enable selective therapy delivery and monitoring in one or more heart chamber;

FIG. 3 is a simplified block diagram of a single monitoring and pacing channel for acquiring pressure, impedance and cardiac EGM signals employed in monitoring cardiac function and/or delivering therapy, including pacing therapy, in accordance with embodiments of the invention;

FIG. 4 is a pictorial representation of the relationship between various types of data that may be analyzed in making a diagnosis decision;

FIG. 5 is a timeline describing portions of an exemplary stored episode;

FIG. 6 is a block diagram of a node of a Bayesian network in accordance with certain embodiments of the invention;

FIG. 7 is a block diagram of several nodes of a Bayesian network in accordance with certain embodiments of the invention;

FIG. 8 is a block diagram of several nodes of a Bayesian network in accordance with certain embodiments of the invention;

FIG. 9 is a block diagram of several nodes of a Bayesian network in accordance with certain embodiments of the invention;

FIG. 10 is a block diagram of several nodes of a Bayesian network in accordance with certain embodiments of the invention;

FIG. 11 is a block diagram of several nodes of a Bayesian network in accordance with certain embodiments of the invention; and

FIG. 12 is a flow chart illustrating a method of analyzing an episode stored by an implantable medical device (IMD) in accordance with certain embodiments of the invention.

### DETAILED DESCRIPTION OF THE INVENTION

The following detailed description of the invention is merely exemplary in nature and is not intended to limit the scope of the invention or the application and uses of the invention as defined in the claims. Furthermore, there is no intention to be bound by any theory presented in the preceding background of the invention or the following detailed description of the invention. The detailed description should be read with reference to the drawing figures, in which like elements in different drawings are numbered identically. The drawings depict selected embodiments and are not intended to limit the scope of the invention.

The analysis of stored episode information retrieved from IMD's can be a challenging and time-consuming process for clinicians/physicians. The stored episode information may provide details that help a clinician/physician identify or classify the condition that resulted in detection of a given stored episode. The ability to accurately classify the patient condition may be refined by the incorporation of information about the specific patient, including arrhythmia history and demographic information. For example, a physician might consider a patient's baseline heart rhythm, previous episode information, the rhythm classification assigned by a device or system (e.g., the implantable detection algorithm), the use of any medications, and/or any classifications assigned by other clinicians or experts. Further refinements may be obtained by incorporating a body of “expert knowledge” in the field, typically comprising statistics regarding various symptoms and diagnoses.

The additional information described above may complicate the analysis of stored episode information, and its use may be limited due to constraints on resources such as time and equipment. Further, the use of such additional information may not always be applied consistently, and may be further prone to subjectivity. Methods and systems in accordance with certain embodiments of the invention may therefore include organizing and accumulating various types of data related to patients with IMDs, and generating information about likely causes and diagnoses based therefrom. Certain embodiments of the invention may include, or may be adapted for use in, diagnostic monitoring equipment, external medical device systems, and implantable medical devices (IMDs), including implantable hemodynamic monitors (IHMs), implantable cardioverter-defibrillators (ICDs), cardiac pacemakers, cardiac resynchronization therapy (CRT) pacing devices, drug delivery devices, or combinations of such devices, and programming systems associated with such devices.

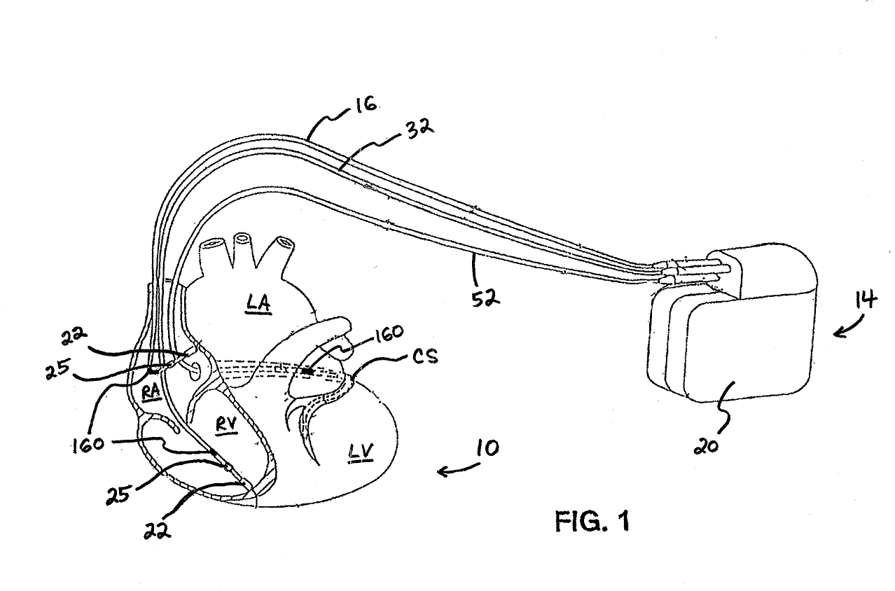

FIG. 1 is a schematic representation of an implantable medical device (IMD) 14 that may be used in accordance with certain embodiments of the invention. The IMD 14 may be any device that is capable of measuring a variety of signals, such as the patient's intra-cardiac electrogram (EGM) signals and/or hemodynamic parameters (e.g., blood pressure signals), for example.

In FIG. 1, heart 10 includes the right atrium (RA), left atrium (LA), right ventricle (RV), left ventricle (LV), and the coronary sinus (CS) extending from the opening in the right atrium laterally around the atria to form the great vein.

FIG. 1 depicts IMD 14 in relation to heart 10. In certain embodiments, IMD 14 may be an implantable, multi-channel cardiac pacemaker that may be used for restoring AV synchronous contractions of the atrial and ventricular chambers and simultaneous or sequential pacing of the right and left ventricles. Three endocardial leads 16, 32 and 52 connect the IMD 14 with the RA, the RV and the LV, respectively. Each lead has at least one electrical conductor and pace/sense electrode, and a can electrode 20 may be formed as part of the outer surface of the housing of the IMD 14. The pace/sense electrodes and can electrode 20 may be selectively employed to provide a number of unipolar and bipolar pace/sense electrode combinations for pacing and sensing functions. The depicted positions in or about the right and left heart chambers are merely exemplary. Moreover, other leads and pace/sense electrodes may be used instead of the depicted leads and pace/sense electrodes. IMD 14 may be an implantable cardioverter defibrillator (ICD), a cardiac resynchronization therapy (CRT) device, an implantable hemodynamic monitor (IHM), a drug delivery device, or any other such device or combination of devices, for example without limitation, according to various embodiments of the invention.

Typically, in cardiac pacing systems of the type illustrated in FIG. 1, the electrodes designated above as “pace/sense” electrodes may be used for both pacing and sensing functions. In addition, some or all of the leads shown in FIG. 1 could carry one or more pressure sensors for measuring systolic and diastolic pressures, and a series of spaced apart impedance sensing leads for deriving volumetric measurements of the expansion and contraction of the RA, LA, RV and LV, according to certain embodiments.

The leads and circuitry described above can be employed to record EGM signals, blood pressure signals, and impedance values over certain time intervals. The recorded data may be periodically telemetered out to a programmer operated by a physician or other healthcare worker in an uplink telemetry transmission during a telemetry session, for example.

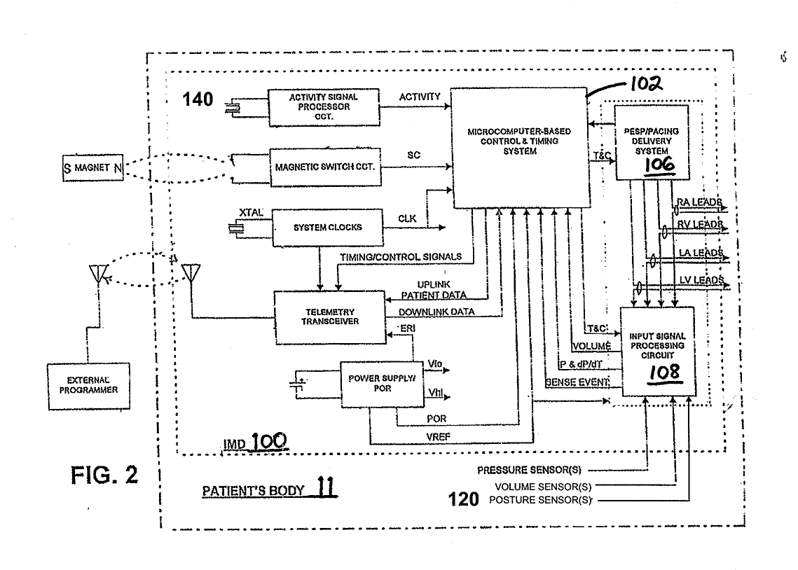

FIG. 2 depicts a system architecture of an exemplary multi-chamber monitor/sensor IMD 100 implanted into a patient's body 11 that provides delivery of a therapy and/or physiologic input signal processing. The typical IMD 100 has a system architecture that is constructed about a microcomputer-based control and timing system 102 which varies in sophistication and complexity depending upon the type and functional features incorporated therein. The functions of microcomputer-based multi-chamber monitor/sensor control and timing system 102 are controlled by firmware and programmed software algorithms stored in RAM and ROM, including PROM and EEPROM, and are carried out using a CPU or ALU of a typical microprocessor core architecture.

The therapy delivery system 106 can be configured to include circuitry for delivering cardioversion/defibrillation shocks and/or cardiac pacing pulses delivered to the heart or cardiomyostimulation to a skeletal muscle wrapped about the heart. Alternately, the therapy delivery system 106 can be configured as a drug pump for delivering drugs into the heart to alleviate heart failure or to operate an implantable heart assist device or pump implanted in patients awaiting a heart transplant operation. The input signal processing circuit 108 includes at least one physiologic sensor signal processing channel for sensing and processing a sensor derived signal from a physiologic sensor located in relation to a heart chamber or elsewhere in the body. Examples illustrated in FIG. 2 include pressure, volume, and posture sensors 120, but could include other physiologic or hemodynamic sensors.

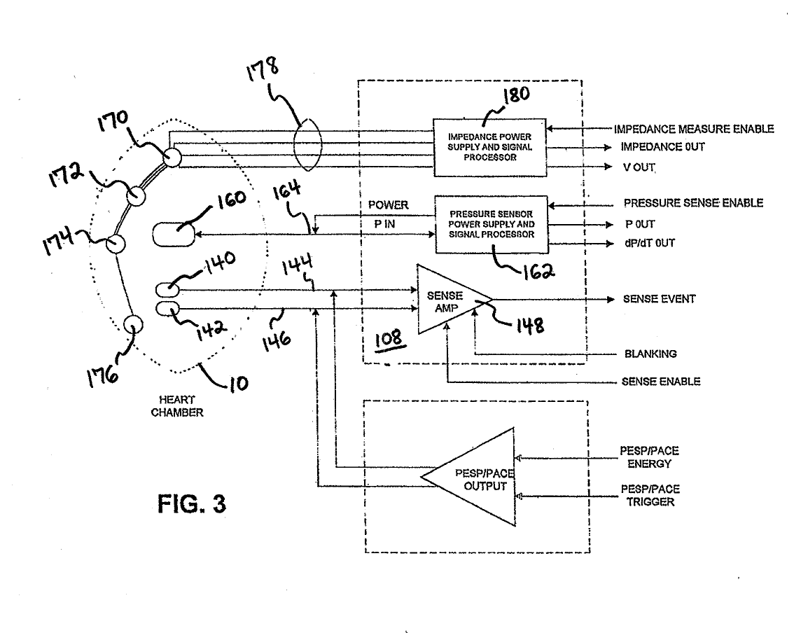

FIG. 3 schematically illustrates one pacing, sensing and parameter measuring channel in relation to one heart chamber. A pair of pace/sense electrodes 140, 142, a pressure sensor 160, and several impedance measuring electrodes 170, 172, 174, 176 are shown located in operative relation to the heart 10. The pair of pace/sense electrodes 140, 142 are located in operative relation to the heart 10 and coupled through lead conductors 144 and 146, respectively, to the inputs of a sense amplifier 148 located within the input signal processing circuit 108. The sense amplifier 148 is enabled during prescribed times when pacing is either enabled or not enabled in a manner known in the pacing art. The sense amplifier provides a sense event signal signifying the contraction of the heart chamber commencing a heart cycle based upon characteristics of the EGM.

The pressure sensor 160 is coupled to a pressure sensor power supply and signal processor 162 within the input signal processing circuit 108 through a set of lead conductors 164. Lead conductors 164 convey power to the pressure sensor 160, and convey sampled blood pressure signals from the pressure sensor 160 to the pressure sensor power supply and signal processor 162. The pressure sensor power supply and signal processor 162 samples the blood pressure impinging upon a transducer surface of the sensor 160 located within the heart chamber when enabled by a pressure sense enable signal from the control and timing system 102. Absolute pressure (P), developed pressure (DP) and pressure rate of change (dP/dt) sample values can be developed by the pressure sensor power supply and signal processor 162 or by the control and timing system 102 for storage and processing.

A variety of hemodynamic parameters may be recorded, for example, including right ventricular (RV) systolic and diastolic pressures (RVSP and RVDP), estimated pulmonary artery diastolic pressure (ePAD), pressure changes with respect to time (dP/dt), heart rate, activity, and temperature. Some parameters may be derived from others, rather than being directly measured. For example, the ePAD parameter may be derived from RV pressures at the moment of pulmonary valve opening, and heart rate may be derived from information in an intracardiac electrogram (EGM) recording.

The set of impedance electrodes 170, 172, 174 and 176 is coupled by a set of conductors 178 and is formed as a lead that is coupled to the impedance power supply and signal processor 180. Impedance-based measurements of cardiac parameters such as stroke volume are known in the art, such as an impedance lead having plural pairs of spaced surface electrodes located within the heart 10. The spaced apart electrodes can also be disposed along impedance leads lodged in cardiac vessels, e.g., the coronary sinus and great vein or attached to the epicardium around the heart chamber. The impedance lead may be combined with the pace/sense and/or pressure sensor bearing lead.

The data stored by IMD 14 may include continuous monitoring of various parameters, for example recording intracardiac EGM data at sampling rates as fast as 256 Hz or faster.

When a patient with an implantable medical device (IMD) experiences a certain type of episode (i.e., the IMD detects a predefined episode), data about the episode may be stored in the IMD. For example, a patient with a cardiac rhythm management (CRM) device, such as an implantable cardioverter defibrillator (ICD), may experience an episode which may be detected by the device, due to a fast ventricular rate, for example, and data about the episode may be stored in the device memory for later retrieval. Stored episode data may include intracardiac electrogram (EGM) signals, marker channel signals, hemodynamic measurements, and a variety of impedance measurements, as an illustrative, but not exhaustive, list of examples of stored data known in the art. An episode may be detected in an ICD, for example, based upon a fast ventricular rate which satisfies certain programmed detection criteria, such as rate and duration criteria. A detected episode may or may not result in therapy (e.g., a defibrillation or cardioversion shock, and/or pacing therapy, and/or drug dispensing therapy) being delivered by the device to the patient. As used herein, the term “CRM device” may be used to encompass at least ICDs, pacemakers, and CRT devices, and any other device which may be adapted to detect the occurrence of a cardiac arrhythmia or cardiac condition of interest, for example.

When the patient next sees a clinician/physician, the stored episode (or episodes) may be retrieved from the IMD memory, for example, via a telemetry download session initiated by a programming system, as is known in the art. The programming system may enable the clinician/physician to observe stored episode data and related stored data, such as EGMs, stored patient information, device programming parameters, and any other signals or measurements captured by the device, for example. The programming system may also retrieve certain episode metrics or measurements calculated by the device, or may be adopted to calculate certain episode metrics from the episode data retrieved from the IMD.

For a given stored episode, the clinician/physician may make a diagnosis based upon evaluation of the stored episode data and related information. The clinician/physician's diagnosis may take into account information about the particular episode, such as episode metrics retrieved or calculated by the programming system. The diagnosis may also take into account information that the clinician/physician is aware of relating to this particular patient, and/or statistical information about patients with similar backgrounds (e.g., similar demographics), and/or information or knowledge in the field (i.e., “domain expert” knowledge) for use in interpreting stored episode information, such as probabilities linking certain types of episode information or episode metrics to likely causes, for example.

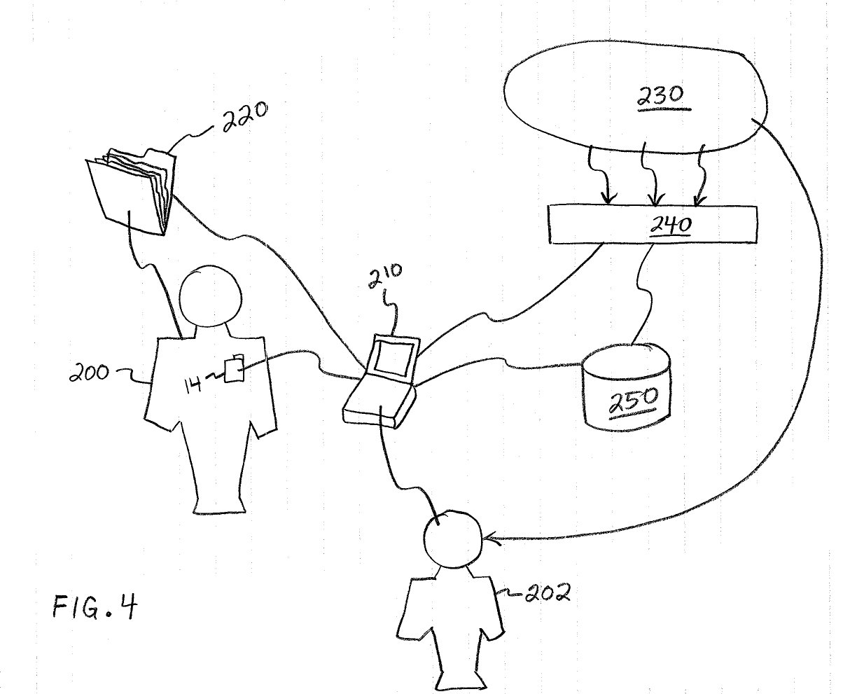

The above scenario is described pictorially in FIG. 4. FIG. 4 shows patient 200 with IMD 14 presenting to clinician/physician 202, for example, at a routine follow-up visit. Clinician 202 may use programmer 210 to communicate with IMD 14 and determine whether any episodes have been detected and stored in IMD 14. If so, programmer 210 may be instructed by clinician 202 to retrieve stored episode information from IMD 14 for analysis and evaluation thereof.

In analyzing stored episode information, clinician 202 may consider not only information specific to a particular episode, but may also likely consider information about the patient's history and/or demographic information, as indicated pictorially by patient information 220. The clinician 202 may further consider information from a body of knowledge collectively referred to as expert domain knowledge 230. Expert domain knowledge 230 may be obtained from a variety of database sources, for example, and communicated electronically to the programmer 210 via a network 240 and/or a number of processors 250, for example. Alternately, or additionally, expert domain knowledge 230 may be communicated to clinician 202 via means such as journal articles and research studies, for example, and provided to programmer 210 manually, according to certain embodiments.

Bayesian networks are a way of incorporating “expert knowledge” into decision-making processes while allowing for uncertainty through the use of probabilities. Expert knowledge may, for example, comprise probabilistic information about symptoms and diagnoses that may inform analysis. For example, a certain “symptom” (or other form of evidence that may be observed during an episode) may be “known” (or believed by experts in the field) to occur with a certain likelihood (probability) if a particular condition (diagnosis) is already known to be present. Thus, when making diagnosis decisions, a likely diagnosis based on Bayesian network analysis may account for such probabilistic information. As used herein, the term “symptom” encompasses not only traditional forms of evidence or symptoms such as those normally reported verbally to a physician by a patient (e.g., dizziness, nausea, headaches, etc.), but is also used to encompass episode metrics (characteristics) associated with particular episodes retrieved from an IMD. For example, whether an ICD patient's ventricular cycle length (VCL) is regular or not may comprise one such episode metric or “symptom” which may be provided by the device and/or determined by a programming system for a given stored episode.

Bayes' theorem can be expressed as a mathematical equation that describes the relationship that exists between simple and conditional probabilities. Bayes' decision theory assumes that a given decision problem (for example, whether an observed episode belongs to one class or another) is posed in probabilistic terms, and that all of the relevant probabilities are known. For instance, the expression P(wᵢ) may be described as the “prior probability” that a certain episode is of the type wᵢ. As an example, P(VT) may denote the prior probability that a given episode is ventricular tachycardia (VT) before the episode is analyzed. The expression p(vₓ|wᵢ) may be described as the “conditional probability” of observing evidence vₓ given the fact that the episode is of a known type or diagnosis, namely type wᵢ. As an example, p(VCL=regular|VT) may denote the conditional probability that the observed ventricular cycle length (VCL) for an episode will be regular (i.e., having a relatively small amount of variability over a number of cycles) given that the episode is known to be VT. In other words, p(vₓ|wᵢ) is a probability density function of non-negative value, which may be estimated by domain experts and/or provided by evaluation of previously collected data (e.g., a statistical database), or by some other suitable means, for example. Next, the expression P(wᵢ|vₓ) may be described as the “posterior probability,” which is the probability (between 0 and 1) that an episode is of a particular type or diagnosis wᵢ given that evidence vₓ is observed. The posterior probability can be calculated from the prior and conditional probabilities, P(wᵢ) and p(vₓ|wᵢ), respectively, according to Bayes' theorem:
P(wᵢ|vₓ)=p(vₓ|wᵢ)*(P(wᵢ)/P(vₓ)). (01)

To continue the above example, the posterior probability that a particular stored episode in an ICD is due to VT, given that the VCL is regular, may be calculated from Eq. (01) as:
P(VT|VCL=regular)=p(VCL=regular|VT)*P(VT)/P(VCL=regular). (02)

A Bayesian network may be a subset of the full joint probability distribution. For instance, if there are 5 binary variables that an expert might consider in making a particular decision, you could describe the full joint probability distribution using 2⁵−1 (or 32−1=31) probabilities. With a Bayesian network, however, if you know, or can assume, that certain variables are independent of other variables, you can reduce the number of probabilities to a much smaller number of probabilities, because you need only specify the prior probabilities for “root nodes,” and the conditional probabilities of non-root nodes given their immediate predecessors.

Eq. (01) is reproduced below, substituting the abbreviations “diag” and “symp” to facilitate the discussion that follows:
P(diag|symp)=P(symp|diag)*P(diag)/P(symp) (03)

Each of the terms on the right side of Eq. (03) is described in more detail below.

The probability of a symptom given a diagnosis, P(symp|diag), can be obtained from domain expert information or knowledge, for example, from statistical or clinical database information and/or from knowledge obtained from experts (e.g., results of clinical studies, estimates of experts). As an example, if it is known that a given stored episode in an ICD is VT, the likelihood that a regular (i.e., stable) ventricular cycle length (VCL) will be observed prior to detection of the episode may be provided by statistical information and/or estimates from those knowledgeable in the field.

For P(diag), the prevalence/incidence of a certain diagnosis in the patient population (e.g., the prior probability of a given diagnosis) is used. For example, in the population of ICD patients, the percentage of all detected episodes that are due to VT may be expressed as a prior probability. Similarly, the percentage of all detected episodes that are due to Atrial Tachycardia (AT) may be expressed as a prior probability. These prior probabilities may be obtained from clinic and database records, for example, or may be estimated by a domain expert, or by the consensus estimate of a number of domain experts. The prior probabilities for all potential diagnoses may be obtained or estimated, for example, including such other diagnoses as sinus tachycardia (ST) and ventricular fibrillation (VF).

For the probabilities of certain symptoms, P(symp), values may be set to their occurrence or prevalence in a given patient population. Alternately, for simplicity, the probabilities may be set to “equal” values (e.g., equally likely). For example, if there are 4 possible values for a given symptom type (e.g., 4 possible episode metric values), the probability of a given symptom, P(symptomᵢ) can be set to 0.25 for each of the 4 possible values. To continue the VCL example, the symptom type “VCL Regularity” could be defined to have 4 possible values, for example: regular, slightly irregular, moderately irregular, and highly irregular. Thus, the probability of each of these 4 symptoms could be set to 0.25 (at least initially) to simplify the analysis.

In certain embodiments, the data may be available to derive the probabilities of certain symptoms, P(symp), from other conditional and prior probabilities. For example:
P(symp)=P(symp|diag1)*P(diag1)+P(symp|diag2)*P(diag2)+ . . . for all diagnoses. This can also be expressed as:

$$P ( symp ) = {\sum }_{i} P ( symp \vert {diag}_{i} ) \times P ( {diag}_{i} )$$

With the respective probabilities obtained from domain experts, clinical databases, and/or observed symptoms, Bayes' theorem may then be applied to compute the probability of each potential diagnosis given the observed symptoms, P(diag|symp), for any stored episode.

The use of Bayesian Network analysis is next described and applied with reference to the accompanying drawing figures in the following example, which describes an implantable cardioverter defibrillator (ICD) patient who presents with stored episodes. The example described is by way of illustration and not limitation; the methods described may be generally applicable to other types of IMD's and other types of diagnosis decisions.

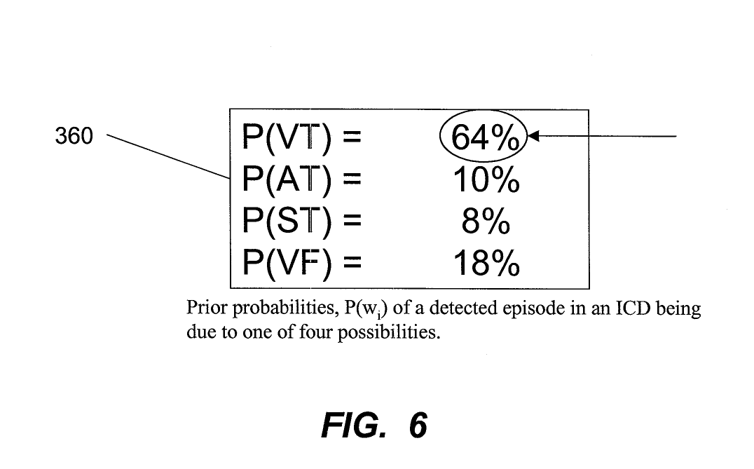

When an ICD detects and stores an arrhythmia episode, a number of potential arrhythmia classifications may be defined as being the possible causes of the episode being detected and/or stored in the device. In the example of a stored episode in an ICD, the list of potential arrhythmia classifications would likely encompass arrhythmias that may result in a fast ventricular rate (e.g., a ventricular rate fast enough to satisfy the detection criterion of the ICD). In the example shown in FIG. 6, four potential arrhythmia classifications are provided, namely AT, VT, VF, and ST. In certain embodiments of the invention, the list of potential arrhythmia classifications could be extended to include other potential causes of a detected episode in an ICD. For example, certain non-physiologic issues such as oversensing, lead problems (e.g., dislodgement, lead fractures, faulty or intermittent lead connections, lead failures), myopotentials (e.g., electrical signals generated by muscle activity), and/or electromagnetic interference or noise (EMI), may also lead to detected episodes in an ICD. Although various embodiments of the invention may include such additional arrhythmia classifications, they have not been included in the examples that follow to facilitate the explanation.

As shown in FIG. 6, and as reproduced in Table I below, each of the four potential arrhythmia classifications is assigned a prior probability value that indicates the likelihood that any given detected episode is a result of each arrhythmia classification. The potential conditions (arrhythmia classifications) and prior probabilities may form a root node 360 of a Bayesian network, according to certain embodiments of the invention. For example, it may be estimated that the likelihood of a detected episode in an ICD being due to VT is 64%, given no other information about the specific patient, or about the particular episode. Similarly, it may be estimated that the likelihood of a detected episode being due to AT is 10%, given no other information about the specific patient, or about the particular episode. Estimates for the likelihood of VF and ST are likewise provided as 18% and 8%, respectively.

TABLE I
Prior Probabilities of each arrhythmia classification

P(rhythm is VT) = 0.64
P(rhythm is AT) = 0.10
P(rhythm is ST) = 0.08
P(rhythm is VF) = 0.18

It should be noted that the estimates of the prior probabilities, such as the examples provided in FIG. 6, may be obtained from statistical analysis of a target patient population, for example, from a database with information about the numbers and types of detected episodes from a large number of ICD patients. Alternately, the estimates could be provided by one or more experts in the field, for example, in the form of published research, study results, or perhaps the consensus estimates of a number of experts in the field.

Based solely on the prior probability information, without any additional information about the episode, or about the particular patient, the most likely classification for a detected episode in an ICD is VT, since it has the highest prior probability, in this case, 64%, as indicated in FIG. 6.

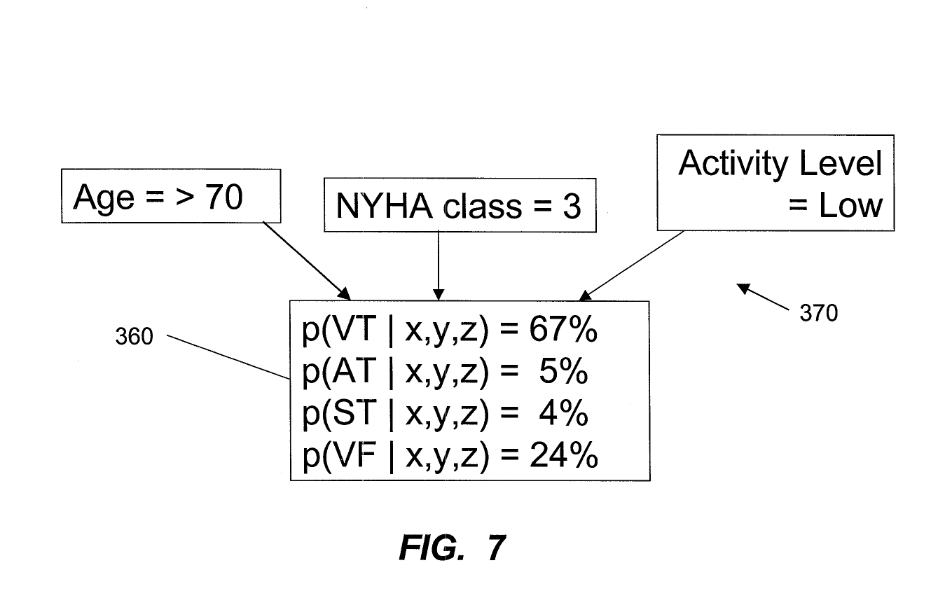

FIG. 7 shows the addition of a number of patient information nodes 370 to form a simple Bayesian network. The patient information nodes include information that is specific to a particular patient, i.e., patient metrics. In the example shown, three patient information nodes 370 are provided, including nodes that describe the patient's age, general activity level, and New York Heart Association (NYHA) category or class. The patient metric associated with each node is described by a discrete value that further identifies the particular patient. In this example, the patient metric for age may denote that the patient is greater than 70 years old, the patient metric for activity level may denote that the patient is relatively inactive, and the patient metric for NYHA category may be class 3. As shown, the information from the patient information nodes 370 may cause the probability of each potential arrhythmia classification to change somewhat. This may be understood as being due to changing the nature of the target patient population. Alternately, the revised probabilities for each arrhythmia classification may be thought of as conditional probabilities for each arrhythmia classification given the information in the preceding nodes.

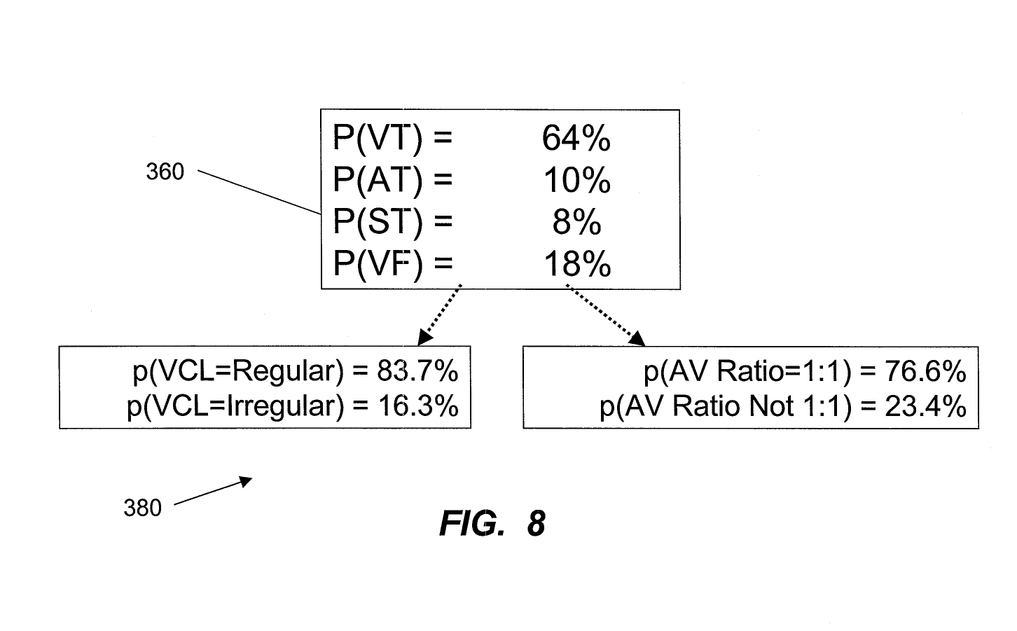

FIG. 8 shows the addition of a number of stored episode information nodes 380 to the root node 360 from FIG. 6 to create a simple Bayesian network. The stored episode information nodes 380 include information that may be specific to a particular episode. In the example shown, two types of stored episode information, or symptoms, are obtained for a particular episode. One such symptom may be described as ventricular cycle length (VCL) Regularity, which may be defined as having two possible values, regular and irregular, according to certain embodiments. Another such symptom may be the atrio-ventricular (AV) Ratio, which may also be defined as having two possible values, for example, One-to-one or Not one-to-one. Of course, many other symptoms may be included to thereby expand the number of stored episode information nodes 380 according to various embodiments of the invention. Furthermore, a symptom may have more than two possible values or episode metrics in various embodiments. However, the examples shown have been simplified to facilitate explanation. Note that the probability information shown in FIG. 8 are prior probabilities at this point.

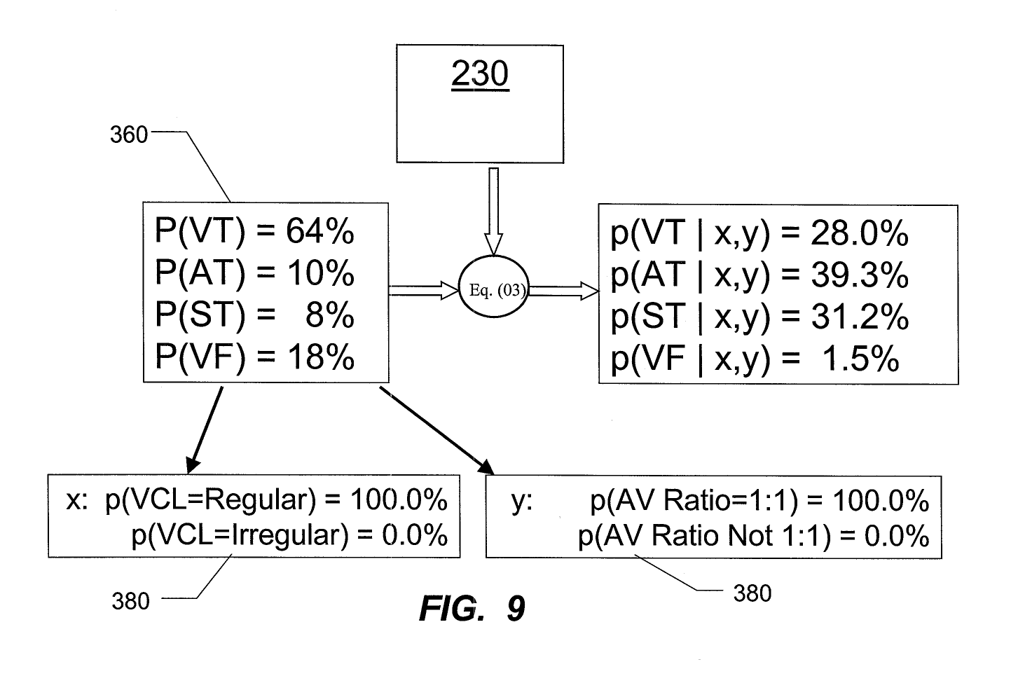

FIG. 9 shows how Bayes' theorem may be applied to incorporate information from the stored episode information nodes 380, as well as information from domain expert information 230, to calculate posterior probabilities for each of the potential arrhythmia classifications. The domain expert information 230 may, for example, include statistics and/or probabilities that relate information regarding observed episode metrics or symptoms to the various potential arrhythmia classifications. For example, the domain expert information may include conditional probabilities that a particular symptom (e.g., VCL=regular) will occur given that a particular arrhythmia classification (e.g., VT) is known. The domain expert information may also include prior probability information for both the arrhythmia classifications and the symptoms, according to certain embodiments of the invention.

The prior probability information from FIG. 8 may next be applied to the domain expert information 230, which includes the conditional probabilities provided below in Table II, to calculate posterior probabilities according to Bayes' theorem. Table II provides conditional probabilities that a certain symptom will occur given a particular diagnosis (e.g., arrhythmia classification) for the ICD patient population:

TABLE II

| Symptom: VCL = Regular or | Symptom: AV Ratio = 1-to-1 or Not |
|---|---|
| Irregular | 1-to-1 |
| P(VCL = Regular \| rhythm | P(AV ratio is 1:1 \| rhythm is VT) = |
| is VT) = 0.85 | 0.10 |
| P(VCL = Regular \| rhythm | P(AV ratio is 1:1 \| rhythm is AT) = |
| is AT) = 0.85 | 0.90 |
| P(VCL = Regular \| rhythm | P(AV ratio is 1:1 \| rhythm is ST) = |
| is ST) = 0.80 | 0.95 |
| P(VCL = Regular \| rhythm | P(AV ratio is 1:1 \| rhythm is VF) = |
| is VF) = 0.80 | 0.02 |

Lastly, as shown in FIG. 9, the symptoms observed for this particular stored episode for this particular patient are: 1) VCL is regular (e.g., 100% likelihood), and 2) AV ratio is 1:1 (e.g., 100% likelihood).

With this information, Eq. (03) can be used to compute the new posterior probabilities. When, as in this case, there is more than one symptom observed for a particular episode, the posterior probability of a diagnosis given the existence of two known symptoms, s1 and s2, may be calculated as follows:
P(Diagnosis|s1,s2)=P(s1,s2|Diagnosis)*P(Diagnosis)/P(s1,s2) (04)

If one can assume that nodes that do not have a path connecting them are independent, then the first term on the right hand side of Eqn. (04) can be expressed as follows:
P(s1,s2|Diagnosis)=p(s1|Diagnosis)*p(s2|Diagnosis) (05)

Equation (04) then becomes:
P(Diagnosis|s1,s2)=p(s1|Diagnosis)*p(s2|Diagnosis)*P(Diagnosis)/P(s1, s2)

Given the probabilities provided by clinical databases and/or domain experts, we can calculate the following for the example in FIG. 9:

$$\begin{matrix}P ( VT \vert ( VCL = reg , AV ratio = 1 \text{:} 1 ) = p ( VCL = reg \vert VT ) * p ( AV ratio = 1 \text{:} 1 \vert VT ) * P ( VT ) / P ( VCL = reg , AV ratio = 1 \text{:} 1 ) = ( 0.85 ) * ( 0.10 ) * ( 0.64 ) / P ( VCL = reg , AV ratio = 1 \text{:} 1 ) = 0.0544 / P ( VCL = reg , AV ratio = 1 \text{:} 1 ) & ( 06 ) \\ P ( AT \vert ( VCL = reg , AV ratio = 1 \text{:} 1 ) = p ( VCL = reg \vert AT ) * p ( AV ratio = 1 \text{:} 1 \vert AT ) * P ( AT ) / P ( VCL = reg , AV ratio = 1 \text{:} 1 ) = ( 0.85 ) * ( 0.90 ) * ( 0.10 ) / P ( VCL = reg , AV ratio = 1 \text{:} 1 ) = 0.0765 / P ( VCL = reg , AV ratio = 1 \text{:} 1 ) & ( 07 ) \\ P ( ST \vert ( VCL = reg , AV ratio = 1 \text{:} 1 ) = p ( VCL = reg \vert ST ) * p ( AV ratio = 1 \text{:} 1 \vert ST ) * P ( ST ) / P ( VCL = reg , AV ratio = 1 \text{:} 1 ) = ( 0.80 ) * ( 0.95 ) * ( 0.08 ) / P ( VCL = reg , AV ratio = 1 \text{:} 1 ) = 0.0608 / P ( VCL = reg , AV ratio = 1 \text{:} 1 ) & ( 08 ) \\ P ( VF \vert ( VCL = reg , AV ratio = 1 \text{:} 1 ) = p ( VCL = reg \vert VF ) * p ( AV ratio = 1 \text{:} 1 \vert VF ) * P ( VF ) / P ( VCL = reg , AV ratio = 1 \text{:} 1 ) = ( 0.80 ) * ( 0.02 ) * ( 0.18 ) / P ( VCL = reg , AV ratio = 1 \text{:} 1 ) = 0.0029 / P ( VCL = reg , AV ratio = 1 \text{:} 1 ) & ( 09 )\end{matrix}$$

Since our example assumes that there are only four possible arrhythmia classifications, the sum of the probabilities must equal one:
P(VT|(VCL=reg, AV ratio=1:1)+P(AT|(VCL=reg, AV ratio=1:1)+P(ST|(VCL=reg, AV ratio=1:1)+P(VF|(VCL=reg, AV ratio=1:1)=1.

Substituting the above calculated values:
0.0544/P(VCL=reg, AV ratio=1:1)+0.0765/P(VCL=reg, AV ratio=1:1)+0.0608/P(VCL=reg, AV ratio=1:1)+0.0029/P(VCL=reg, AV ratio=1:1)=1,
and therefore
P(VCL=reg, AV ratio=1:1)=0.0544+0.0765+0.0608+0.0029=0.1946.

Substituting this value back into equations (06)-(09) yields the following posterior probabilities:
P(VT|VCL=reg, AV ratio=1:1)=0.0544/0.1946=0.280
P(AT|VCL=reg, AV ratio=1:1)=0.0765/0.1946=0.393
P(ST|VCL=reg, AV ratio=1:1)=0.0608/0.1946=0.312
P(VF|VCL=reg, AV ratio=1:1)=0.0029/0.1946=0.015

FIG. 9 shows the updated values for the likelihood of each of the potential arrhythmia classifications (e.g., the posterior probability for each arrhythmia classification) given the two observed symptoms. Note that the posterior probability values for AT and ST have both increased from their previous values, while the posterior probability values for VT and VF have both decreased, due to the nature of the observed symptoms. The most likely arrhythmia classification is now AT, with a likelihood of 39.3%.

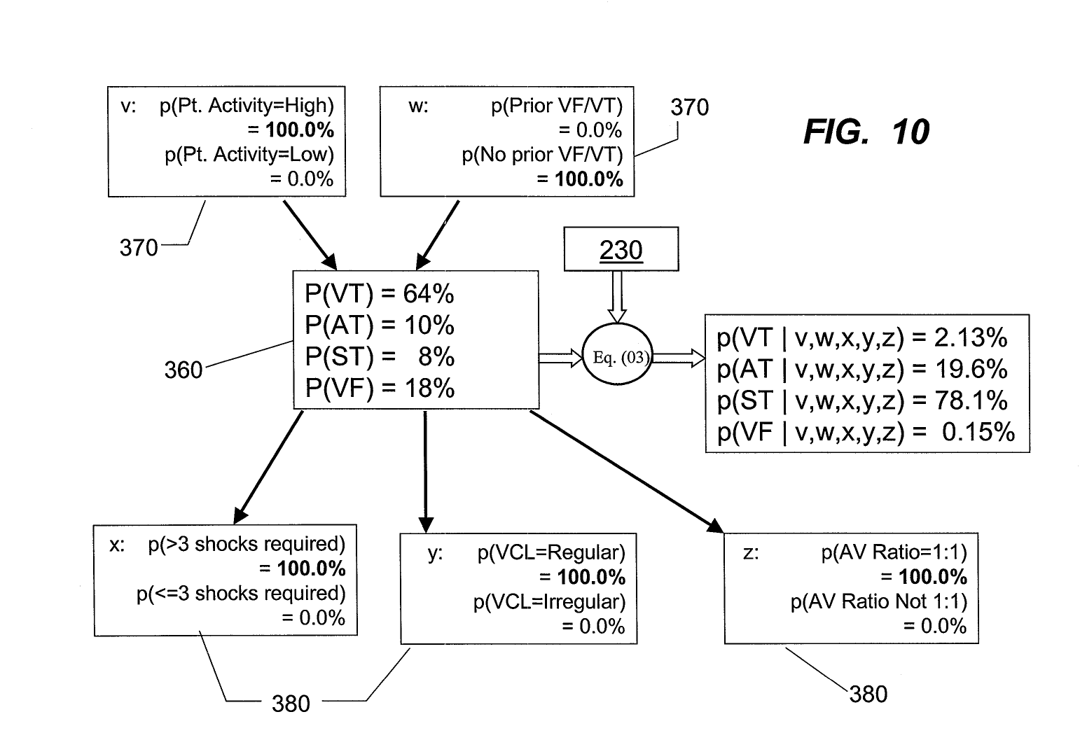

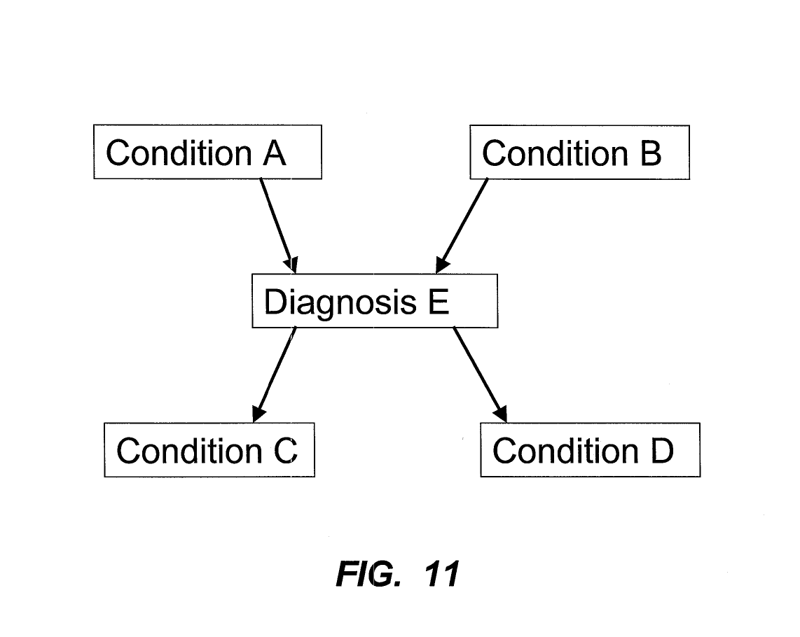

To complete the illustration, an example is provided in FIG. 10 in which a root node 360 has both stored episode information nodes 380 and patient information nodes 370. As shown, patient activity level and prior history of VTNF have been added as patient information nodes, with patient activity level “high” and “no” prior history of VT/VF entered as the patient metrics (a scenario which might occur in a primary prevention type ICD patient, for example). As one might surmise, this additional information may affect the likelihood of a given episode being due to a particular type of arrhythmia. The analysis is similar to that described above, using the prior probabilities of the upper nodes, and the conditional probabilities of each set of descendant nodes given their immediate predecessors. (This technique is described in general terms with respect to FIG. 11, below.) As shown, the most likely classification for the episode is now ST (78.1% likely).

In the generalized example illustrated in FIG. 11, two conditions in the top node, A and B, are known, as are two conditions in the bottom nodes, C and D. The diagnosis, E, may be expressed using Bayes' theorem as a function of the prior and conditional probabilities of the four nodes with respect to the diagnosis, E, as follows:
P(e|abcd)=P(abcd|e)*P(e)/P(abcd). (10)

Eq. (10) can be expressed through a series of steps as:
P(e|abcd)=(P(e)*P(ab|e)/P(ab))*P(cd|e)/(P(cd|ab)).

As described in the above examples, we can obtain values for P(e), P(ab|e), p(ab), p(cd|e) from domain experts, and then set the sum of P(e|abcd) for all possible values of e equal to 1, to solve for P(cd|ab). That value can then be substituted into the equation above to solve for the individual values of P(e|abcd) for all the possible values of e.

In certain embodiments of the invention, a method of classifying a stored arrhythmia episode may include retrieving stored episode information from an implantable medical device (IMD). Retrieving stored episode information from an IMD may be performed, for example, using a programming system, as is known in the art. The stored episode information may include certain metrics that describe the episode, or certain aspects of the episode. These episode metrics may be referred to as symptoms or observed evidence. Examples of episode metrics include the above mentioned ventricular cycle length and AV ratio. Examples of symptoms or observed evidence associated with these particular episode metrics may, for example, include VCL=regular, and AV ratio=1-to-1. The episode metrics may have symptoms associated with them that are binary in nature, or may have three or more possible values, for example. AV ratio, for example, may be extended to have three possible symptoms by further dividing the symptom “Not 1-to-1” into two symptoms such as “A greater than V,” and “V greater than A.”

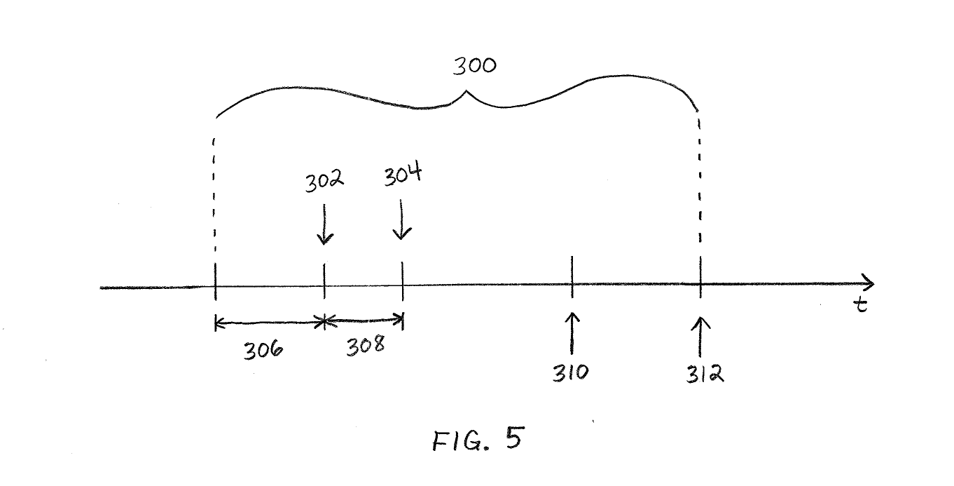

It should be noted that in analyzing stored episode information, the episode metrics or symptoms, such as those described above, may be determined from various portions of the stored episode, as shown in FIG. 5. FIG. 5 illustrates a timeline describing portions of an exemplary stored episode 300. Stored episode 300 may be due, for example, to an arrhythmia that begins at onset 302, which may be triggered by a measured rate parameter exceeding a predetermined threshold, for example. In certain embodiments, detection 304 may occur only if the condition that triggered onset 302 is maintained for a predetermined period, illustrated as duration 308. In certain embodiments, a certain amount of pre-episode information may also be stored as part of stored episode 300, as indicated by pre-episode buffer 306. Other portions of stored episode 300 may also be defined, such as therapy delivery 310 and episode termination 312, for example. Thus, the calculation or derivation of episode metrics or symptoms may be based on stored episode information from certain pre-determined portions of the episode, such as during the duration 308 interval, or from the interval between detection 304 and therapy delivery 310, or between therapy delivery 300 and episode termination 312, or from any other similar interval that may be defined within stored episode 300.

In certain further embodiments of the invention, retrieving stored episode information may include retrieving information from episodes that occurred prior to the one being analyzed. Such prior episode information may be obtained from the IMD, or may be obtained from a programming system memory, or may be provided via access to a network, for example. Prior episode information may be used, for example, to update the domain expert information (e.g., to change the probabilities linking symptoms and causes). Prior episode information may also be used in certain embodiments of the invention to affect the inputs to the patient information nodes. For example, a patient information node may include a metric that indicates whether the patient has ever had a previous VT or VF episode. Alternately, certain embodiments may include morphology template matching using known VT episodes to confirm that the episode being analyzed is VT. For example, a morphology template based on stored electrogram (EGM) data from a known VT episode may be used to compare to the episode being analyzed, either as a confirmation step, or to derive an additional episode metric therefrom. The use of prior episode information to update the domain expert information may further include the application of weighting factors in certain embodiments, for example, to give greater weight to more recent prior episodes, or to weight certain types of episodes according to severity.

Certain embodiments of the invention may allow the system to “learn” as new information becomes available. For example, domain expert information may be initialized by loading a database of information (including episode data, for example), then periodically updating the domain expert information via downloads from a network connection, which may aggregate data from a large number of patients from sources such as clinical, research, and/or registration databases. Such updates to domain expert information may include prior probabilities and conditional probabilities, as well as patient demographic data. Certain further embodiments of the invention may include additional ways to update domain expert information, such as by allowing manual inputting of data by a clinician, perhaps based on research results and/or published studies, or even perhaps allowing a certain level of customization based on user preference, for example.

Certain embodiments of the invention may include the ability to add to or modify aspects of the relationships between various nodes, the nodes representing diagnoses, episode information, and patient information. For example, independence relationships and/or causal relationships may be introduced or changed in a Bayesian network to thereby reduce the full joint probability distribution to a Bayesian network representing a smaller subset that may be defined by independent and dependent relationships.

A method or system in accordance with certain embodiments of the invention may, after retrieving stored episode information, produce a list of potential diagnoses, along with an indication (e.g., a probability) of each potential diagnosis being correct. The probabilities may be determined automatically upon retrieval of the stored episode information, using Bayesian network analysis as described herein. In certain further embodiments, the most likely diagnosis may be provided by determining which of the potential diagnoses has the highest probability of being correct. In other embodiments, the most likely diagnosis may be provided only if the probability associated therewith exceeds some predetermined threshold, for example, to avoid producing a result that is less than 50% likely. In still further embodiments, the probabilities of a number of potential diagnoses may be provided along with suggestions for obtaining additional information that may lead to a more definitive result.

It should be noted that the example provided is a relatively simple example of a Bayesian network. The ideas presented in the above examples may be extended to cover much more complex network node structures. For example, many additional types of episode characteristics (e.g., symptoms) may be employed.

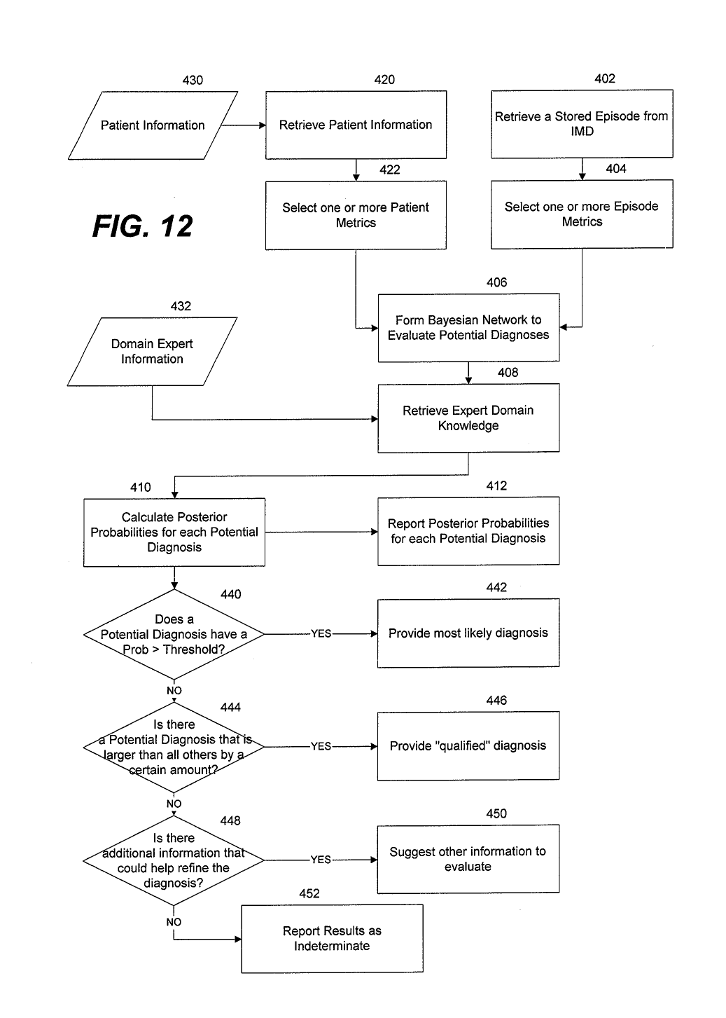

FIG. 12 is a flow chart describing a method of analyzing episodes stored by an implantable medical device (IMD) according to an exemplary embodiment of the invention. The method may, in some embodiments, be initiated by a physician command, such as during a patient follow-up visit. Alternately, the analysis may be performed automatically based on data collected (e.g., by the IMD) or other suitable triggers. Data collection may include automatic uploads of data, for example, from patient at-home monitors or from instruments in clinics.

The method described in FIG. 12 may, for example, be performed by a suitable processor programmed with instructions to perform the method, according to various embodiments of the invention. For example, the posterior probabilities and other outcomes may be computed on a remote computer or server, such as at a hospital site or other customer site. Alternately, the processing may take place at a centralized location, for example, at a manufacturer's site, where the manufacturer may provide the analysis as a service to a number of customer or client sites. This embodiment could be facilitated by communications and data transfer via the Internet in certain embodiments.

In other embodiments, a programmer or programming system may be adapted to perform methods in accordance with embodiments of the invention. For example, a programmer may periodically receive updated domain expert information, either automatically (e.g., via the Internet), or manually (e.g., via updates to programmer software or memory). Thus, a programmer may be used to retrieve stored episode information from an IMD, then analyze stored episodes using domain expert information available to the programmer, for example. In still further embodiments, an IMD may also perform methods according to embodiments of the invention, and may make the analysis available via a programming system, or via a remote monitoring system, or other suitable means.

A method of analyzing stored episodes may include the following steps, described with continued reference to FIG. 12. As a possible first step, step 402 may include retrieving a stored episode from the IMD, for example, using telemetry communication between a programmer and the IMD as is known in the art. The stored episode information retrieved in step 402 may include one or more episode metrics, each of which describes a condition or symptoms observed in conjunction with the particular episode. Step 404 may be performed next, involving the selection of one or more episode metrics to consider in analyzing the stored episode. Step 406 may be performed next in certain embodiments to form a Bayesian network that represents relationships between episode metrics selected in step 404 and one or more potential diagnoses. Domain expert information may next be retrieved, as indicated by step 408. As discussed above, domain expert information may come from a variety of sources, including clinical and research databases and/or published studies, as well as less formal sources. Step 410 may next calculate posterior probabilities for one or more potential diagnoses, for example, by applying Bayes' theorem to the domain expert information and episode metrics. The resulting posterior probabilities may next be reported to a clinician/physician according to step 412 as shown, to facilitate a diagnosis decision.

The method described FIG. 12 may optionally include the following steps to assist the clinician/physician in making a diagnosis decision. For example, certain embodiments of the invention may include the use of a threshold value to select a most likely diagnosis from a plurality of potential diagnoses. This may be accomplished as shown at steps 440 and 442, which determine whether of potential diagnosis has a posterior probability that exceeds the threshold value, and if so, reports the potential diagnoses to the clinician/physician. In certain embodiments, the method may alternately or additionally include a step such as step 444 in FIG. 12, which determines whether a potential diagnosis exists which has a posterior probability larger than the other potential diagnoses by a certain amount, for example. If so, step 446 may provide this diagnosis to a clinician/physician. The method may, for example, “qualify” the diagnosis so provided to indicate to the clinician/physician that the result may be less reliable than that produced by step 442. It should be noted that step 444 could be defined to include simply selecting the potential diagnosis that has the highest posterior probability, if one exists.

Certain embodiments of the invention may further address the situation where none of the potential diagnoses either exceed a predefined threshold or sufficiently exceed the other potential diagnoses. For example, step 448 determines whether additional information, if known, could help inform the diagnosis decision. If such information exists, step 450 may provide suggestions to the clinician/physician about which types of information to seek can evaluate in order to make the diagnosis decision. Lastly, if it is determined that no such additional information may be helpful to the diagnosis, an indeterminate result may be reported to the clinician/physician without further comment.

The method illustrated in FIG. 12 also includes steps for incorporating patient-specific information into the analysis described above. For example, step 420 includes retrieving patient information. Step 420 may encompass a number of ways of acquiring patient-specific information, such as manual entry of information provided verbally by the patient, retrieval of information about the patient stored in the IMD, retrieval of information about the patient stored in a programming system, and information retrieved from a network database system, to name just a few possible sources. One or more patient metrics may next be selected, as shown at step 442, for inclusion in the analysis. A patient metric may, for example, include demographic information, such as age or gender, and may also encompass historical information about the patient, such as whether they have ever previously experienced certain types of episodes.

In certain embodiments of the invention, patient information and/or domain expert information may be updated as new information becomes available. For example, FIG. 12 represents patient information 430 and domain expert information 432 as data sources, which may change over time as new information is provided to update these data sources. In certain embodiments of the invention, the information used to provide updates to patient information 430 and domain expert information 432 may be selected by a user/clinician/physician as deemed appropriate. It should be noted that in certain cases, some types of patient metrics may change as a result of previously analyzed stored episodes. One example of this might occur where a patient metric is defined to indicate whether a patient has previously experienced a certain type of episode (e.g., a particular diagnosis). Once a stored episode occurs in which that particular type of episode is identified, the patient metric would need to be updated to reflect the change in the patient's episode history.

Thus, APPARATUS AND METHODS FOR ARRHYTHMIA EPISODE CLASSIFICATION have been provided. While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A method of analyzing an episode stored by an implantable medical device (IMD), comprising:
retrieving information about a stored episode from an IMD, the stored episode information including an episode metric;
retrieving domain expert information about one or more potential diagnoses and one or more episode metrics; and
applying the domain expert information to the episode metric to determine a likelihood that the stored episode is indicative of a particular potential diagnosis.

2. The method of claim 1 further comprising retrieving patient information including at least one patient metric.

3. The method of claim 2 wherein the patient metric identifies patient demographic information.

4. The method of claim 2 wherein the patient metric comprises historical patient information.

5. The method of claim 4 wherein the historical patient information includes information about previous stored episodes.

6. The method of claim 1 wherein domain expert information includes prior probability and conditional probability information describing the relationship between episode metrics and potential diagnoses.

7. The method of claim 6 wherein the domain expert information further includes conditional probability information describing a likelihood of one or more episode metrics being observed if a particular potential diagnosis is known to be present.

8. The method of claim 6 wherein the domain expert information further includes prior probability and conditional probability information describing the relationship between patient information and potential diagnoses.

9. The method of claim 1 wherein the potential diagnoses include both physiologic and non-physiologic causes.

10. The method of claim 9 wherein the non-physiologic causes include at least one of oversensing, electromagnetic interference (EMI), lead malfunctions, and myopotentials.

11. The method of claim 1 wherein the step of applying domain expert information to determine the likelihood that the stored episode was due to one or more potential diagnoses further comprises applying Bayes' theorem to calculate posterior probabilities of one or more of the potential diagnoses.

12. The method of claim 1 further comprising identifying a likely diagnosis by selecting the potential diagnosis with the highest posterior probability.

13. The method of claim 1 further comprising identifying a likely diagnosis by selecting the potential diagnosis that exceeds a predetermined threshold.

14. A computer-readable medium programmed with instructions for performing a method of analyzing an episode stored by an implantable medical device (IMD), the medium comprising instructions for causing a programmable processor to:
retrieve information about a stored episode from an IMD, the stored episode information including an episode metric;
retrieve domain expert information about one or more potential diagnoses and one or more episode metrics; and
apply the domain expert information to the episode metric to determine a likelihood that the stored episode was due to a particular potential diagnosis.

15. The medium of claim 14 further comprising instructions to retrieve patient information including at least one patient metric.

16. The medium of claim 14 wherein the IMD is a cardiac rhythm management (CRM) device, and wherein potential diagnoses include cardiac arrhythmia classifications.

17. The medium of claim 14 further comprising instructions to include non-physiologic causes among the potential diagnoses.

18. The medium of claim 14 further comprising instructions to apply Bayes' theorem to calculate posterior probabilities of one or more of the potential diagnoses using the following equation:
P(D₁|S₁, S₂)=P(S₁, S₂|D₁)*[P(D₁)/P(S₁, S₂)],
where P(S₁, S₂|D₁) is the conditional probability that episode metrics S₁ and S₂ will both be observed if the diagnosis is known to be D₁, and
where P(D₁) and P(S₁, S₂) are the prior probabilities of D₁ occurring, and of both S₁ and S₂ occurring, respectively.

19. The medium of claim 14 further comprising instructions to identify a likely diagnosis by selecting the potential diagnosis with the highest posterior probability.

20. The medium of claim 14 further comprising instructions to identify a likely diagnosis by selecting the potential diagnosis that exceeds a predetermined threshold.

21. A system for analyzing episodes stored by an implantable medical device (IMD), the system comprising:
retrieval means for retrieving stored episodes from an IMD, the stored episodes including one or more episode metrics;
informational retrieval means for retrieving domain expert information about one or more potential diagnoses and one or more episode metrics; and a processor for determining a likelihood that a given stored episode was due to a particular diagnosis, wherein information retrieval means is adapted to receive domain expert information automatically via a network, and wherein the processor is adapted to determine the likelihood of a particular diagnosis by calculating a posterior probability according to the equation:
P(D₁|S₁, S₂)=P(S₁, S₂|D₁)*[P(D₁)/P(S₁, S₂)],
wherein P(S₁, S₂|D₁) is the conditional probability that episode metrics S₁ and S₂ will both be observed if the diagnosis is known to be D₁, and wherein P(D₁) and P(S₁, S₂) are the prior probabilities of D₁ occurring, and of both S1 and S₂ occurring, respectively.

22. The system of claim 21 further adapted to allow manual modification of the domain expert information.

23. The system of claim 21 wherein the domain expert information may be modified based on episode information from previously stored episodes.

24. The system of claim 21 wherein the retrieval means for retrieving stored episodes from an IMD includes a programmer.

25. The system of claim 21 wherein the retrieval means for retrieving stored episodes from an IMD includes a computer network.
